(19) United States
(12) Patent Application Publication
(10) Pub. No.: US 2016/0243233 A1
(43) Pub. Date: Aug. 25, 2016

(54) MANAGEMENT OF TRACTIONAL MEMBRANES

(71) Applicant: MARK HUMAYUN, Glendale, CA (US)

(72) Inventor: MARK HUMAYUN, Glendale, CA (US)

(21) Appl. No.: 14/918,078

(22) Filed: Oct. 20, 2015

Related U.S. Application Data
(63) Continuation of application No. 14/218,850, filed on Mar. 18, 2014, now abandoned.
(60) Provisional application No. 61/801,941, filed on Mar. 15, 2013.

(51) Int. Cl. A61K 41/00; A61K 38/48; A61K 9/00

(52) U.S. Cl. CPC A61K 41/0047; A61K 9/0048; A61K 38/484; C12Y 304/21007

(57) ABSTRACT

Methods and compositions are provided here to manage tractional membranes, as well as other diseases and disorders of the eye. Compositions comprising microbubbles associated with enzymes or vitreolytic agents, are provided. The method of the present invention involves administration of the microbubble and an enzyme or vitreolytic agent to a subject in need thereof, followed by application of ultrasound.

### CROSS REFERENCE TO RELATED APPLICATION

This application claims the benefit of U.S. Provisional Application No. 61/801,941, filed on Mar. 15, 2013 which is incorporated herein by reference in its entirety.

### FIELD OF THE INVENTION

The invention generally relates to compositions and methods for treating diseases and disorders of the eye, such as fractional membranes. The composition of the present invention comprises microbubbles in association with an enzyme or vitreolytic agent. The methods of treatment involve administering a microbubble composition and an enzyme or vitreolytic agent to a subject in need thereof, followed by application of ultrasound. Optionally, the enzyme of vitreolytic agent is associated with the microbubbles.

### BACKGROUND OF THE INVENTION

The vitreous gel (or simply vitreous) of the eye may play an important role in the pathogenesis of various retinal disorders. As the eye ages, it is common for the vitreous to separate from the retina. But if this separation is not complete, the adhesion can create pulling forces on the retina that may result in subsequent loss or distortion of vision. If the disruption to visual acuity is severe enough, it requires treatment.

The current standard of care for treating symptomatic vitreomacular adhesion (VMA) is pars plana vitrectomy (PPV), which involves surgically removing the vitreous from the eye. Recovery is extensive and often requires patient to remain immobilized “head down” for 7-14 days and be reliant on caregivers.

Pharmacologic agents have been used to liquefy the vitreous and complete vitreoretinal separation. A potential vitreolytic agent should be able to facilitate either liquefaction of the vitreous or weakening of the VMA, or ideally both.

A number of enzymes have been the subject of preclinical and clinical evaluations for use as vitreolytic agents, including chondroitinase, collagenase, dispase, hyaluronidase, nattokinase, plasmin, plasminogen activators and ocriplasmin, (JETREA®, ThromboGenics Inc, generic name for microplasmin).

Plasmin is not available for clinical use and is unstable. Therefore, clinical application requires activation of its proenzyme, plasminogen, which in turn is also not commercially available for human use. Ocriplasmin (microplasmin) is a genetically engineered version of plasmin and has been shown in clinical trials to be able to safely release vitreomacular adhesion and close Stage 2 macular holes in a significant number of patients. Microplasmin is now FDA-approved for use in relieving traction in patients with vitreomacular fraction. However, the success rate (26%) for the treatment group is not appreciably higher than 10% in the placebo group (Girach Expert Rev Ophthalmol. 2012; 7(4):311-323; Stalmans, P. et al., N Engl J Med. 2012; 367:606-615).

What is therefore needed are new treatments for symptomatic VMA that have improved efficacy with good safety profile and reduced cost.

### SUMMARY OF THE INVENTION

The present invention relates to compositions and methods for treating diseases and disorders of the eye, such as tractional membranes.

In a first aspect, the present invention is a composition comprising microbubbles, wherein the microbubbles are associated with an enzyme.

In one embodiment, the enzyme is a proteolytic enzyme.

In one embodiment, the enzyme is selected from the group consisting of chondroitinase, collagenase, dispase, hyaluronidase, nattokinase, plasmin, plasminogen activators and ocriplasmin (microplasmin) or combinations thereof. In a particular embodiment, the enzyme is selected from plasmin, microplasmin, hyaluronidase or combinations thereof. In a particular embodiment, the enzyme is microplasmin.

In a particular embodiment, the enzyme is loaded on the microbubble surface.

In another particular embodiment, the enzyme is contained within the microbubble.

In a second aspect, the present invention is a composition comprising microbubbles, wherein the microbubbles are associated with a vitreolytic agent.

In one embodiment, the vitreolytic agent is enzymatic. In another embodiment, the vitreolytic agent is non-enzymatic.

In a particular embodiment, the vitreolytic agent is loaded on the microbubble surface. In another particular embodiment, the vitreolytic agent is contained within the microbubble.

In certain embodiments, the composition of the present invention is suitable for direct injection into the eye.

In a third aspect, the present invention is method of treating a disease or disorder of the eye in a subject in need thereof, comprising (i) administering a composition comprising microbubbles, wherein the microbubbles are associated with an enzyme and (ii) applying ultrasound energy to the eye to activate the microbubbles, thereby releasing the enzyme to treat the disease or disorder.

In one embodiment, the disease or disorder of the eye is a tractional membrane.

In another particular embodiment, the disease or disorder of the eye is a macular hole.

In one embodiment, the enzyme is a proteolytic enzyme.

In a particular embodiment, the enzyme is elected from the group consisting of chondroitinase, collagenase, dispase, hyaluronidase, nattokinase, plasmin, plasminogen activators and ocriplasmin (microplasmin) or combinations thereof.

In an even more particular embodiment, the enzyme is microplasmin.

In a fourth aspect, the present invention is a method of treating a disease or disorder of the eye in a subject in need thereof comprising (i) administering a microbubble composition and an enzyme (iii) applying ultrasound to the eye, thereby treating the disease or disorder of the eye. According to this embodiment of the invention, the microbubble composition and the enzyme may be administered in any order, e.g., the microbubble composition may be administered prior to or concurrently with the enzyme. In certain embodiment, the microbubble composition and the enzyme is administered directly to the eye.

In one embodiment, the disease or disorder is a tractional membrane.

In another embodiment, the disease or disorder is a macular hole.

In one embodiment, the enzyme is a proteolytic enzyme.

In a particular embodiment, the enzyme is elected from the group consisting of chondroitinase, collagenase, dispase, hyaluronidase, nattokinase, plasmin, plasminogen activators and ocriplasmin (microplasmin) or combinations thereof.

In an even more particular embodiment, the enzyme is microplasmin.

In a fifth aspect, the present invention is method of treating a vitreo-pathology in a subject in need thereof, comprising (i) administering a composition comprising microbubbles, wherein the microbubbles are associated with a vitreolytic agent and (ii) applying ultrasound energy to the eye to activate the microbubbles, thereby releasing the vitreolytic agent to treat vitreo-pathology.

In one embodiment, the vitreo-pathology is a vitreo-retinoapathy.

In another embodiment, the vitreo-pathology is a vitreo-maculopathy.

In one embodiment, the vitreolytic agent is an enzymatic vitreolytic agent.

In another embodiment, the vitreolytic agent is a non-enzymatic vitreolytic agent.

In yet another embodiment, the vitreolytic agent is a liquefactant.

In a still further embodiment. The vitreolytic agent is a interfactant.

The dose of the administered may vary. In certain embodiments, the dose of the microbubble composition is effective to provide a dose of the enzyme or vitreolytic agent of between about 10 μg and about 300 μg. In a particular embodiment the dose of the composition is effective to provide a dose of the enzyme or vitreolytic agent of about 100 μg. In a particular embodiment, the dose of the microbubble composition is effective to provide a dose of the enzyme or vitreolytic agent of about 50 μg.

In other embodiment, the dose of the enzyme or vitreolytic agent administered with the microbubble composition is between about 10 μg and about 300 μg. In a particular embodiment the dose of the enzyme or vitreolytic agent is about 100 μg. In a particular embodiment, the dose of the enzyme or vitreolytic agent of about 50 μg.

In a sixth aspect, the present invention is a method of achieving pharmacologic vitreolysis by administered a microbubble composition and an enzyme or vitreolytic agent, wherein the enzyme or vitreolytic agent is optionally associated with the microbubbles.

In a particular embodiment, the method results in vitreolysis without retinal detachment.

In certain embodiments, the microbubble composition is administered by injection in the vitreous cavity of the eye. In a particular embodiment, the microbubble composition is injected in the midvitreous cavity of the eye using the pars plana approach.

In certain embodiments, the ultrasound is applied to the inferior conjunctival surface with ultrasound gel as an acoustic window. In a particular embodiment, the an ultrasound probe having about an 8 MHz frequency and about a 0.25 mechanical index is applied to the inferior conjunctival surface with ultrasound gel as an acoustic window.

### BRIEF DESCRIPTION OF THE DRAWINGS

FIG. 1 provides a bar graph showing the efficacy of plasmin microbubbles on posterior vitreous detachment (PVD) and retinal detachment.

FIG. 2 provides a photomicrograph showing a red-free image of the right eye of a rabbit treated with microbubble(MB) and ultrasound (Group A). Note the normal retinal architecture and vasculature.

FIG. 3 provides a photomicrograph showing optical coherence tomography (OCT) of the right eye of the same rabbit as FIG. 2 treated with MB and ultrasound. Note the lack of PVD but normal retinal architecture.

FIG. 4 provides a photomicrograph showing red-free and fluorescein angiography images of the right eye of a rabbit treated with ¼ dose plasmin (100 ug/100 ul) (Group B). Note the normal retinal architecture and vasculature. FIG. 5 provides an optical coherence tomography (OCT) scan of the right eye of the same rabbit as FIG. 4 treated with ¼ dose plasmin (100 ug/100 ul). Note the lack of PVS with normal retinal architecture.

FIG. 6 provides photomicrographs showing a red-free and fluorescein angiography images of the right eye of a rabbit treated with full dose plasmin (500 ug/100 ul) (Group C). Note the large area of retinal necrosis with unmasking of the choroidal vasculature.

FIG. 7 provides an optical coherence tomography (OCT) scan of the right eye of the same rabbit as FIG. 6 treated with full dose plasmin (500 ug/100 ul). Note the extensive retinal necrosis and flap of detached retina floating into the vitreous cavity.

FIG. 8 provides photomicrographs showing a red-free and fluorescein angiography images of the right eye of a rabbit treated with plasmin (100 ug/ul), MB and ultrasound (Group D). Note the normal retinal architecture and vasculature.

FIG. 9 provides an optical coherence tomography (OCT) scan of the right eye of the same rabbit as FIG. 8 treated with plasmin (100 ug/ul), MB and ultrasound. Note the evident PVD.

FIG. 10 provides an optical coherence tomography scan in an eye with the plasmin+MB treatment showing posterior vitreous detachment 2 days after the primary procedure.

FIG. 11 provides an ultrasound-B scan of an eye treated with plasmin (100 ug/ul), MB and ultrasound showing PVD 2 days after the primary procedure.

FIG. 12 provides an ultrasound-B scan of an eye treated with plasmin (100 ug/ul), MB and ultrasound showing PVD 2 days after the primary procedure.

FIG. 13 provides a scanning electron micrograph image of an eye treated with plasmin (50 ug)/MB/ultrasound showing smooth retinal surface (no adherent vitreous) 1 week after the primary procedure.

FIG. 14 provides a scanning electron micrograph image of an eye treated with plasmin (50 ug)/MB/ultrasound showing completely detached posterior vitreous 1 week after the primary procedure.

FIG. 15 provides an A/B Mode ultrasound image showing the bolus of MB in the vitreous cavity right after the intravitreal injection.

FIG. 16 provides an A/B Mode ultrasound image showing the bolus of MB (circled by dashed lines) in the vitreous cavity after the intravitreal injection at the beginning and end of the experiment.

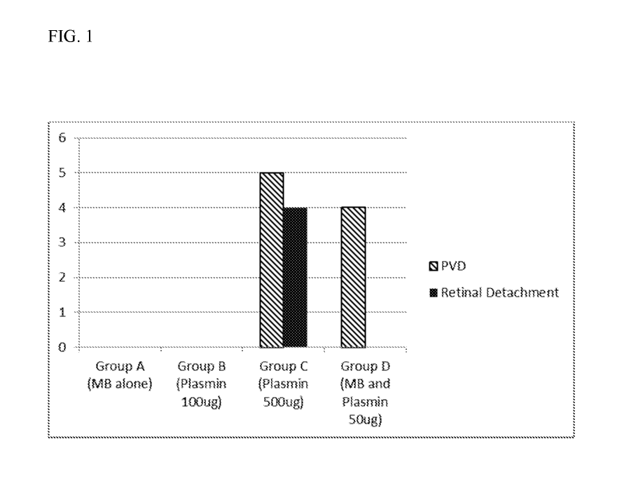

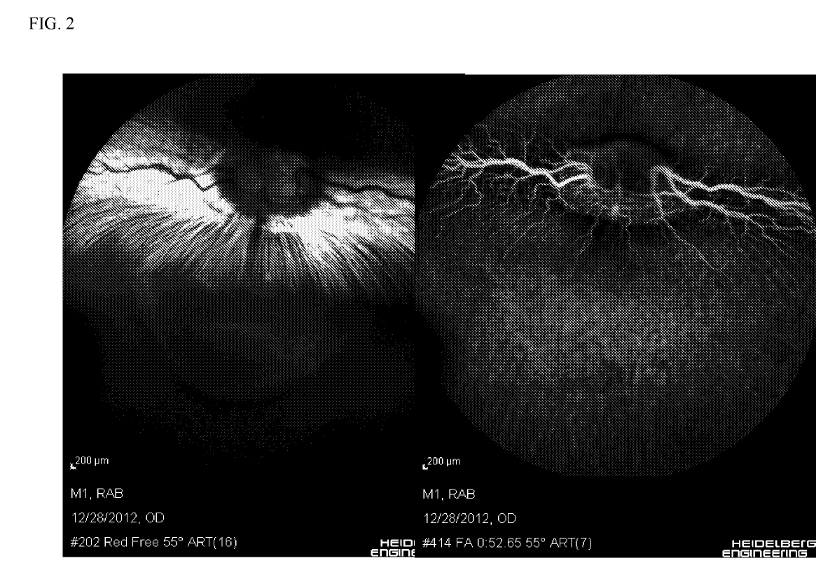

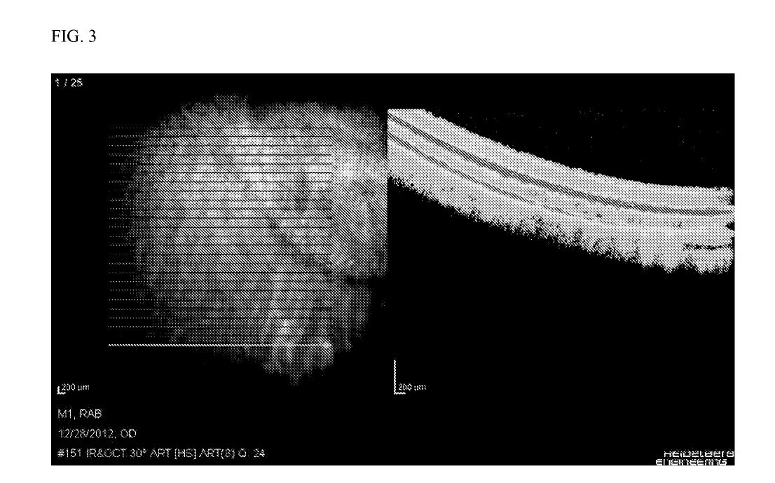

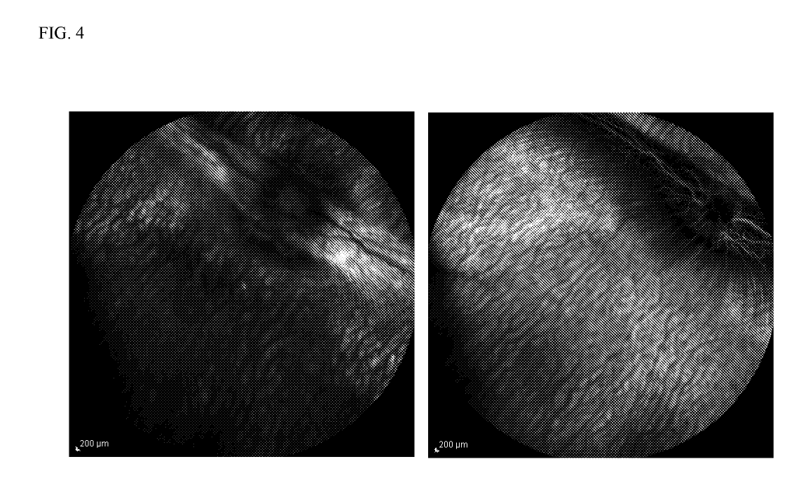

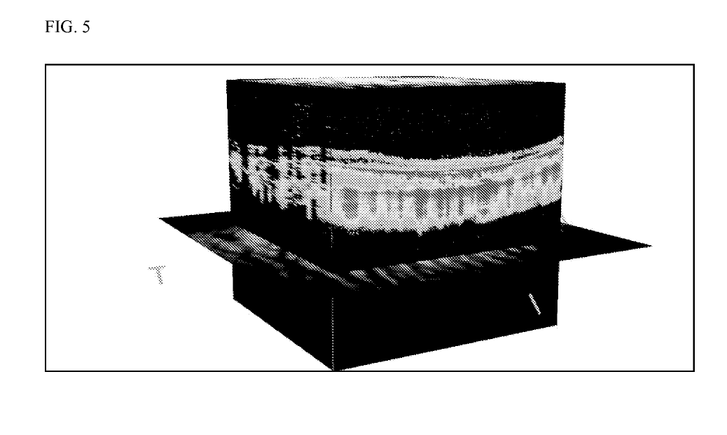

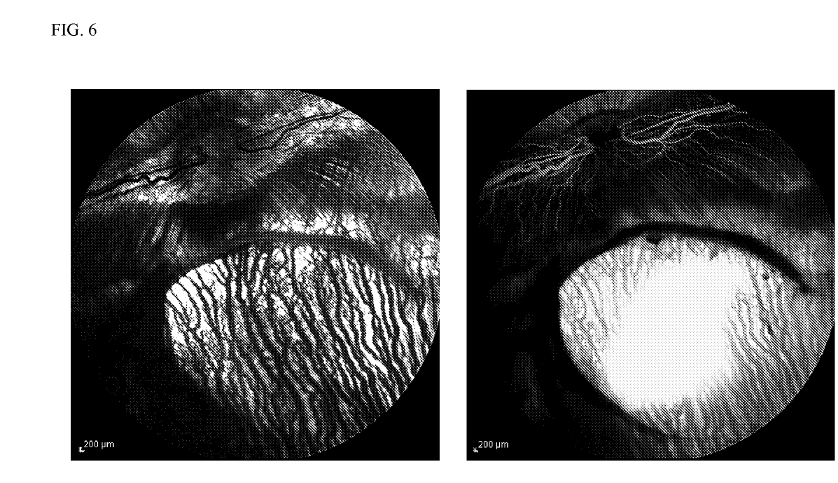

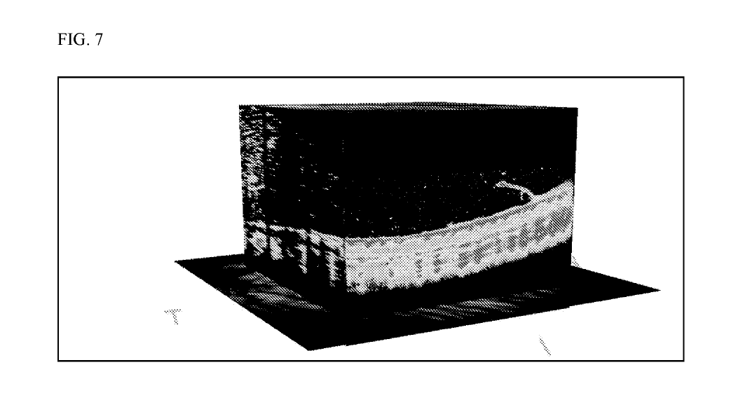

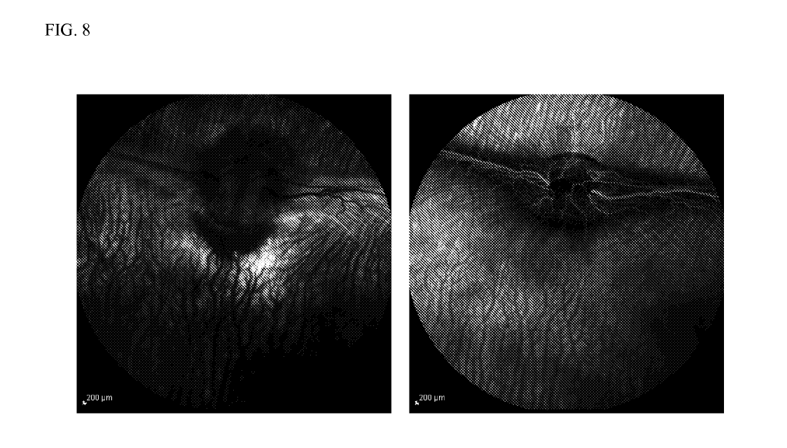

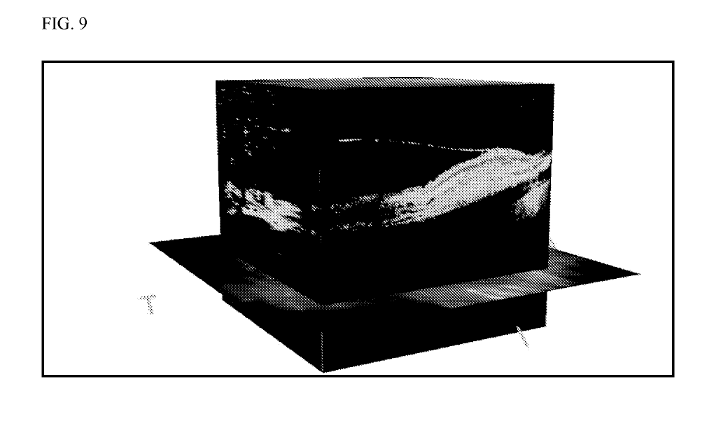

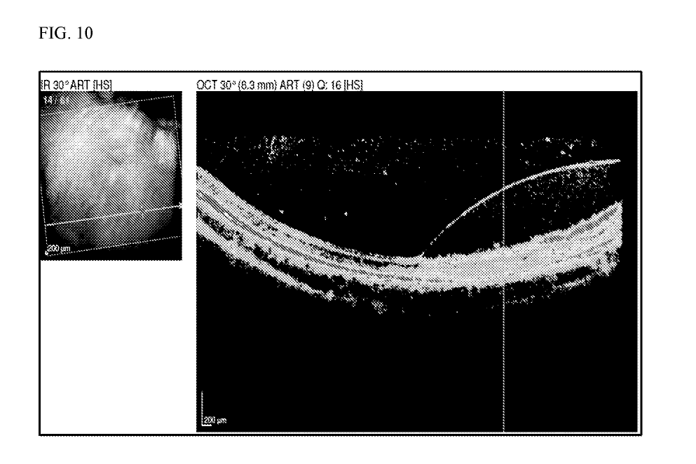

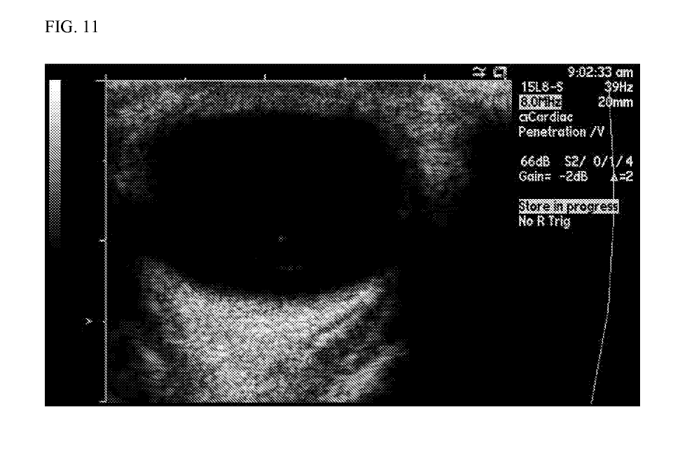

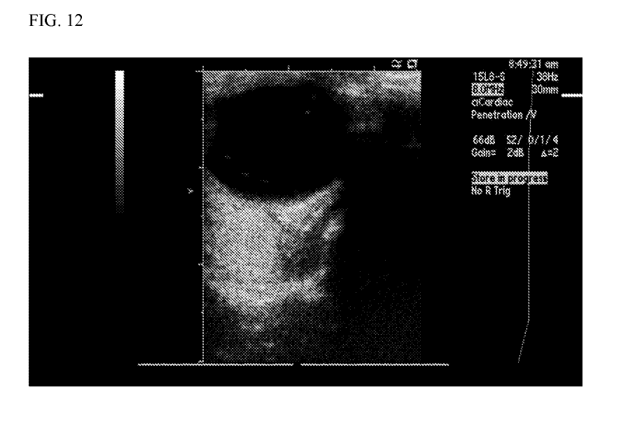

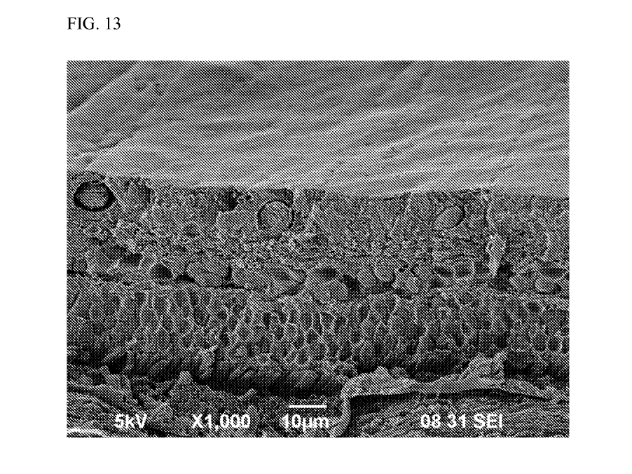

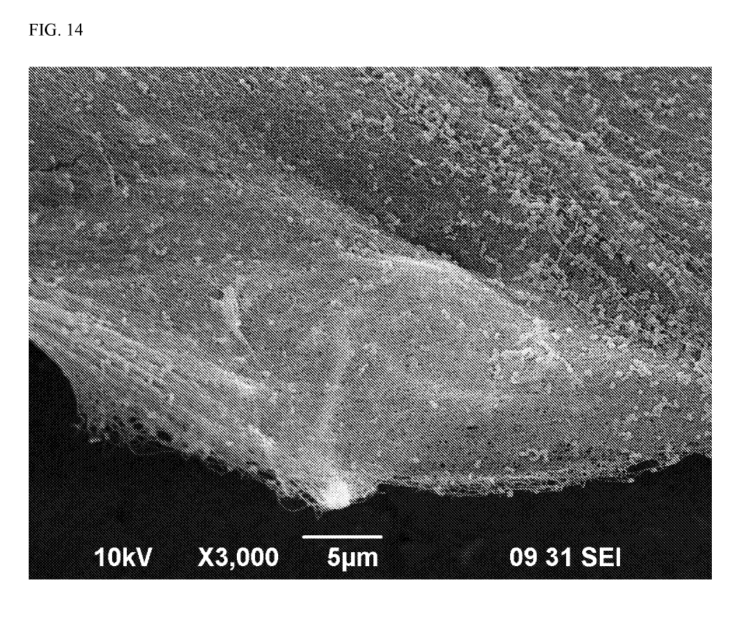

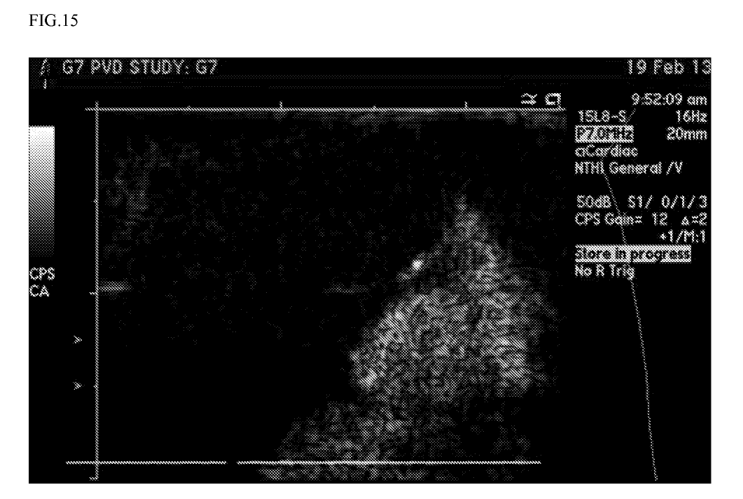

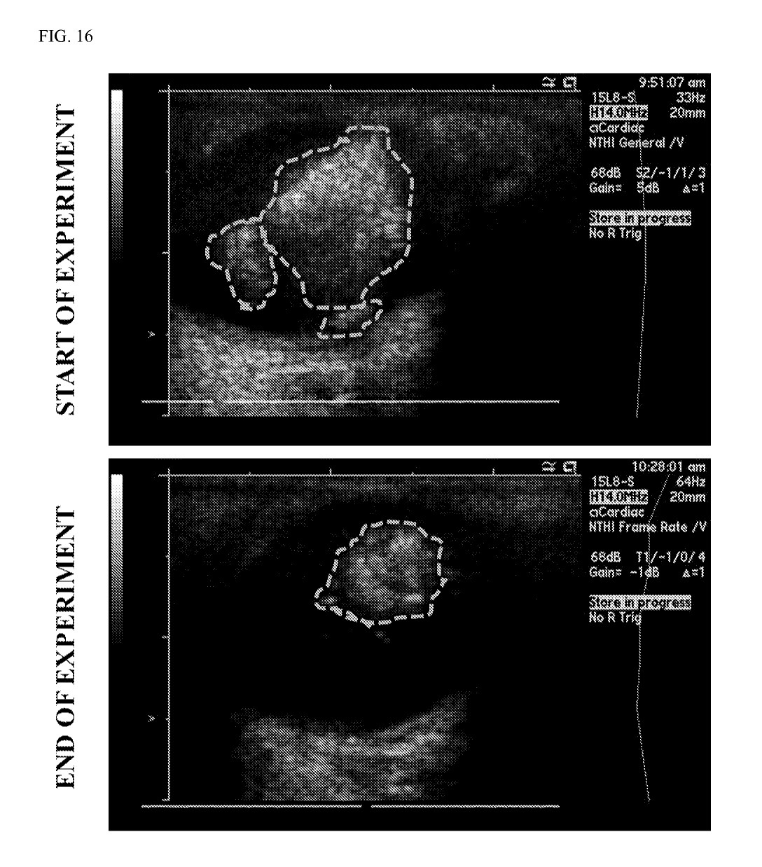

### DETAILED DESCRIPTION OF THE DRAWINGS

Methods and compositions are provided here to treat diseases or disorders of the eye, such as treating tractional membranes. Methods of preparing such compositions are also provided.

The methods described herein advantageously deliver an therapeutic agent, such as an enzyme or vitreolytic agent, to the vitreous more uniformly and homogeneously than the simple intravitreal injection of enzyme. In some instances, this approach can provide a success rate of 100% to relieve vitreomacular traction and induce pharmacologic vitreolysis compared with 26% normally observed for the intravitreal injection of plasmin alone.

The methods described herein advantageously use a lower dose of plasmin that methods known in the art. In one embodiment, the methods described herein use approximately a quarter (and potentially even less) of the plasmin dose needed to relieve vitreomacular traction in similar patients. As a result, they are more efficient and also result in cost savings to the patient and healthcare payer compared to using the full dose. Using a lower dose of plasmin also improves retinal safety issues such as occurrence of retinal detachment as compared to administration of a full dose of plasmin thereby improving the safety profile of this therapy.

Microbubbles

In one aspect, the present invention provides a composition comprising microbubbles, useful in the treatment of diseases and disorders of the eye, such as tractional membranes. Optionally, the microbubbles are associated with a therapeutic agent, such as an enzyme or vitreolytic agent.

Microbubbles are tiny, gas-filled bubbles having a solid or semi-solid shell that can be injected into the bloodstream or vitreous where they remain inactive unless stimulated. They are smaller than one millimeter in diameter, but larger than one micrometer.

Due to the compressible gas core, microbubbles may provide a sensitive acoustic response They are used in medical diagnostics as a contrast agent for ultrasound imaging. The gas-filled microbubbles oscillate and vibrate when a sonic energy field is applied and may reflect ultrasound waves. This distinguishes the microbubbles from surrounding tissues. In practice, because gas bubbles in liquid lack stability and would therefore quickly dissolve, microbubbles must be encapsulated with a solid or semi-solid shell. In various embodiments, the shell is made from a lipid, fat or protein.

Simulation of the microbubbles according to the present invention involves the application of ultrasonic energy or ultrasound. High-frequency ultrasound has been used to create images of bone, tissue and other structures within the body by measuring the speed and intensity with which sound waves bounce off these objects and return as an echo. Ultrasound waves directed at microbubbles cause them to vibrate and return a unique echo within the bloodstream that produces a dramatic distinction, or high “contrast,” between blood vessels and surrounding tissue, thus enabling clinicians to visualize areas of restricted blood flow. Specialized Doppler ultrasound, which measures the rate and volume of blood flow, can further pinpoint the extent and severity of blockage caused by blood clots. Microbubbles may also have therapeutic applications and may be filled or associated with therapeutic agents. Methods and apparati for preparing and using microbubbles are described in patent application WO/2009/020994.

Various microbubbles may be used in the present methods to effect management of tractional membranes. The microspheres are stable and small enough to pass through the pulmonary capillaries. In a particular embodiment, the microspheres are about 1 to about 5 microns in diameter.

In one embodiment, the composition of the present invention comprises microbubbles filled with insoluble perfluorocarbon gas, such as but not limited to, perfluoromethane, perfluoroethane, perfluoropropane, perfluorobutane, perfluoropentane, or a combination thereof, optionally, associated with a therapeutic agent, such as an enzyme (e.g., a proteolytic enzyme) or vitreolytic agent.

In a particular embodiment, the composition comprises microbubbles, wherein the microbubble is a lipid-coated microsphere filled with octafluoropropane gas and is optionally associated with a therapeutic agent, such as an enzyme (e.g., a proteolytic enzyme) or vitreolytic agent. The enzyme may be any suitable enzyme, including but not limited to the enzymes disclosed herein.

In one particular embodiment, the microbubbles are DEFINITY® microbubbles (Lantheus Medical Imaging, Inc). DEFINITY® microbubbles are lipid-coated microspheres filled with octafluoropropane gas. DEFINITY® does not contain any human or animal materials. The microspheres contained in DEFINITY® have a mean diameter of 1.1 to 3.3 microns (in vitro measurements). Optionally, the DEFINITY® microbubble is coated, filled or otherwise associated with a therapeutic agent, such an enzyme (e.g., a proteolytic agent) or a vitreolytic agent. The enzyme may be any suitable enzyme, including but not limited to the enzymes disclosed herein

In another embodiment, the present invention is a composition comprising microbubbles, wherein the microbubble is a perflouropropane gas encapsulated by a serum albumin shell and optionally associated with an enzyme (e.g., a proteolytic enzyme) or a vitreolytic agent. The enzyme may be any suitable enzyme, including but not limited to the enzymes disclosed herein.

In a particular embodiment, the microbubbles are OPTISON® microbubbles (GE Healthcare, Inc.) which comprise perfluoropropane gas encapsulated by a serum albumin shell. Optionally, the OPTISON® microbubble is coated, filled or otherwise associated with an enzyme (e.g., a protelytic enzyme) or a vitreolytic agent. The enzyme may be any suitable enzyme, including but not limited to the enzymes disclosed herein.

In certain embodiments of the composition, the microbubbles are associated with more than one therapeutic agent. For example, the microbubbles are associated with more than one enzyme or vitreolytic agent.

In certain embodiments of the composition, the microbubbles are not unassociated with an enzyme, vitreolytic agent or other therapeutic agent.

In certain embodiment, the microbubbles comprises a single layer. In other embodiment, the microbubbles are multi-layered. A representative, non-limiting multiple layer microbubble comprises a gas core, a polymer and lipid shell surrounding the gas core and a surfactant layer surrounding shell.

In certain embodiments, the microbubbles are targeted. For example, by attaching a targeting ligand, such as a polysaccharide, monoclonal antibody or peptide, to the shell.

Therapeutic Agents

In one embodiment, the microbubbles associated with one or more therapeutic agents, such as an enzyme (e.g., a proteolytic enzyme) or a vitreolytic agent. The application of ultrasonic shock waves can activate the microbubbles associated with the therapeutic agents, causing release of the therapeutic agent. For example, application of ultrasound may result in mini-ruptures to release the therapeutic agent. Associating the microbubbles with a therapeutic agent, optionally visualizing their presence at the diseased site using the ultrasound diagnostic mode, and then activating the microbubbles to release their contents at the targeted lesion/region can be a powerful and effective way to administer a therapeutic agent uniformly, and homogeneously without harming other areas of the eye or body.

As used herein to describe “association” with a microbubble, the term “association” means to be physically in contact with inside or on the surface of a microbubble such that the therapeutic agent is carried by the microbubble into the appropriate intraocular location. The therapeutic agent(s) may be associated with the microbubble by coating on the microbubble surface or being contained (loaded) within the microbubble. In one embodiment, the enzymes are non-covalently joined within or on the surface of or embedded within the wall of the microbubble.

In a particular embodiment, the therapeutic agent is associated with the microbubble by binding to the microbubble shell and through attachment of site-specific ligand.

All or nearly all of the microbubbles may be associated with the therapeutic agent. Alternatively, only a portion of the microbubbles may be associated with the therapeutic agent.

Therapeutic agent-associated microbubbles may be administered to an appropriate site within the body such as the vitreous where they may be subsequently activated with ultrasound applied to the appropriate region of the eye to effect activation.

In one embodiment, the microbubbles are associated with an enzyme, such as a proteolytic enzyme.

Representative, non-limiting examples of enzymes suitable for use in the compositions or methods of the present invention are chondroitinase, collagenase, dispase, hyaluronidase, nattokinase, plasmin, plasminogen activators and ocriplasmin, (ThromboGenics Inc) (aka microplasmin). Variants and derivatives of these enzymes are also suitable for use.

Plasmin, also known as fibrinolysin or lysofibrin, is a serine-type protease which results from the activation of the zymogen plasminogen. Plasmin carries a heavy chain comprising 5 kringle domains and a light chain comprising the catalytic domain. Plasminogen can be enriched from blood plasma, e.g., via lysine affinity-chromatography (Deutsch & Mertz, 1970, Science 170, 1095-1096). Truncation of the plasmin molecule (outside and/or inside the plasmin catalytic domain) is possible as long as the catalytic domain remains functional, such truncation thus results in the formation of a “proteolytically active derivative” of plasmin. As such, one or more of the 5 kringle domains can be deleted wholly or partially.

Truncated plasmins lacking one or more kringle domains and/or lacking parts of one or more kringle domains are suitable for use in the compositions and methods of the present invention. Examples of truncated variants of plasmin include, but are not limited to, midiplasmin, miniplasmin, microplasmin, and delta-plasmin.

Microplasmin has been approved for VMA resolution. The drug's enzymatic properties target the architectural components of the vitreous and the adhesion at the vitreoretinal interface, both of which are implicated in the pathogenesis of symptomatic VMA. Microplasmin exerts proteolytic effects on collagen, fibronectin, and laminin to produce vitreous liquefaction and its detachment from the macula (Gandorfer A., et al., Invest Ophthalmol Vis Sci. 2004; 45:641-7; Sebag J, Green W R. 2012, Vitreous and the vitreo-retinal interface. In: Ryan S J, ed. Retina. 5th ed. Oxford, England: Elsevier; 2012). In multiple preclinical models, intravitreal injection of microplasmin consistently and rapidly (within 30 minutes) induced complete vitreous detachment. These experimental results confirmed its potential viability as a pharmacologic treatment of symptomatic VMA.

In clinical pharmacokinetic studies, microplasmin was undetectable in the eye within 7 days after a single intravitreal injection. Microplasmin administered intravitreally does not result in detectable levels in the systemic circulation. In studies assessing systemic exposure after intravenous administration in humans, microplasmin was inactivated quickly (within seconds) by α2-antiplasmin. The inactive ocriplasmin/α2-antiplasmin complex is then cleared from the circulation with a half-life of several hours. Statistical analysis of the combined data demonstrated VMA resolution in 26.5% of microplasmin-injected patients compared with 10.1% in placebo (p <0.001) (Girach et al., Expert Rev Ophthalmol. 2012; 7(4):311-323). Total posterior vitreous detachment was more prevalent in microplasmin-injected patients (13.4%) compared with placebo-injected patients (3.7%; p <0.001). The methods provided herein offer a success rate of 100% to relieve traction and induce pharmacologic vitreolysis compared with 26% for the microplasmin injection alone.

The combination of ultrasound and microbubbles with microplasmin surprisingly reduces the required dose of enzyme for therapeutic benefit. In clinical trials intravitreal microplasmin alone was administered at doses of 25-175 μg. The 125-μg dose was associated with optimal efficacy in the studies with no additional benefit observed with the 175-μg dose or up to 3 monthly injections of 125 μg. The present methods are effective using reduced doses of microplasmin at about 25% of the full dose used in the clinical trials. Effective doses of microplasmin can be reduced when microplasmin is administered together with microbubbles (either at the same time or in associated with microbubbles). In one embodiment, the effective dose of microplasmin is between about 1 and 50 μg. In one embodiment, the effective dose is between about 20-40 μg and in a particular embodiment, the effective dose of microplasmin is about 30 μg. In another particular embodiment, the effective dose of microplasmin is about 40 μg.

Thus, in one embodiment, the present invention is a composition comprising microbubbles, wherein the microbubbles are associated with an enzyme selected from the group consisting of chondroitinase, collagenase, dispase, hyaluronidase, nattokinase, plasmin, plasminogen activators or ocriplasmin (microplasmin), alone or in combination. In certain embodiments, the enzyme is a variant or derivative of chondroitinase, collagenase, dispase, hyaluronidase, nattokinase, plasmin, plasminogen activators or ocriplasmin (microplasmin).

In a particular embodiment, the composition comprises microbubbles comprising lipid-coated microsphere filled with octafluoropropane gas (e.g., DEFINITY®), wherein the microbubbles are associated with an enzyme, such as proteolytic enzyme.

In a particular embodiment, the composition comprises microbubbles comprising lipid-coated microsphere filled with octafluoropropane gas (e.g., DEFINITY®), wherein the microbubbles are associated with an enzyme selected from the group consisting of chondroitinase, collagenase, dispase, hyaluronidase, nattokinase, plasmin, plasminogen activators or ocriplasmin (microplasmin), alone or in combination, including variants or derivatives thereof.

In another particular embodiment, the composition comprises microbubbles comprising a perflouropropane gas encapsulated by a serum albumin shell (e.g., OPTISON®), wherein the microbubbles are associated with an enzyme, such as a proteolytic enzyme.

In one embodiment, the composition that comprises microbubbles comprising a perflouropropane gas encapsulated by a serum albumin shell (e.g., OPTISON®), wherein the microbubbles are associated with an enzyme selected from the group consisting of chondroitinase, collagenase, dispase, hyaluronidase, nattokinase, plasmin, plasminogen activators or ocriplasmin (microplasmin), alone or in combination, including variants or derivatives thereof.

In another particular embodiment, the present invention is a composition comprising microbubbles, wherein the microbubbles are associated with microplasmin, or derivatives or variations thereo.

In a particular embodiment, the present invention is a composition comprising microbubbles, wherein the microbubbles are coated or filled with microplasmin, or derivatives or variants thereof.

In one embodiment, the present invention is a composition that comprises microbubbles comprising lipid-coated microsphere filled with octafluoropropane gas (e.g., DEFINITY®) wherein the microbubbles are associated with microplasmin.

In one embodiment, the present invention is a composition that comprises microbubbles comprising a perflouropropane gas encapsulated by a serum albumin shell (e.g., OPTISON®) wherein the microbubbles are associated with microplasmin.

In additional the enzyme(s), the microbubbles may be associated with one or more additional therapeutic agents. Representative, non-limiting therapeutic agents include, but are not limited to, anticoagulants, antithrombolytic agent, antiinflammatory agents, antiviral agents, antibacterial agents, antifungal agents, anti-angiogenic agent, anti-mitotic agents, antihistamines or anesthetics. The therapeutic agent may be, for example, and inorganic compound, biological polymer, peptide, polypeptide, antibody, peptide conjugate, nucleic acid, oligonucleotide, polynucleotide, ribozyme, or small interfering RNA (siRNA) capable of rendering a beneficial physiological effect in treating a pathological condition.

In one embodiment, the compositions of the present invention comprises microbubbles in association with a vitreolytic agent.

In one embodiment, the vitreolytic agent is enzymatic.

In a particular embodiment, the enzymatic vitreolytic agent is a non-specific. In a particular embodiment, the non-specific enzymatic vitreolytic agent is selected from the group consisting of tissue plasminogen activator, plasmin, microplasmin or nattokinase.

In another particular embodiment, the enzymatic vitreolytic agent is a substrate-specific. In a particular embodiment, the substrate-specific enzymatic vitreolytic agent is selected from the group consisting of chondroitinase, dispase or hyaluronidase.

In another embodiment, the vitreolytic agent is non-enzymatic. In a particular embodiment, the non-enzymatic vitreolytic agent is urea.

In another particular embodiment, the non-enzymatic vitreolytic agent is an RGD peptide.

In a further embodiment, the vitreolytic agent is a liquefactant. As used herein, the term “liquefactant” means that the vitreolytic agent liquefies the vitreous. Liquefactants may be non-specific or substrate specific. Examples of suitable non-specific liquefacts include, but are not limited to, tissue plasminogen activator, plasmin, microplasmin, nattokinase and vitreosolve. Examples of suitable substate-specific liquifactants include chondroitinase and hyaluronidase

In yet another embodiment, the vitreolytic agent is a an interfactant. As used herein, the term “interfactant” means that the vitreolytic agent alters the vit-ret interace. Interfactants may be non-specific or substrate specific. Examples of suitable non-specific interfactants include, but are not limited to, tissue plasminogen activator, plasmin, microplasmin, nattokinase and vitreosolve. Examples of suitable substrate specific interfactants include dipase, chondroitinase and RGD peptides.

In a particular embodiment, the composition comprises microbubbles comprising lipid-coated microsphere filled with octafluoropropane gas (e.g., DEFINITY®), wherein the microbubbles are associated with a vitreolytic agent, such as enzymatic or non-enzymatic vitreolytic agent.

In another particular embodiment, the composition comprises microbubbles comprising lipid-coated microsphere filled with octafluoropropane gas (e.g., DEFINITY®), wherein the microbubbles are associated a vitreolytic agent, such as a liquefactant or interfactant.

In another particular embodiment, the composition comprises microbubbles comprising a perflouropropane gas encapsulated by a serum albumin shell (e.g., OPTISON®), wherein the microbubbles are associated with vitreolytic agent, such as an enzymatic or non-enzymatic vitreolytic agent.

In one embodiment, the composition that comprises microbubbles comprising a perflouropropane gas encapsulated by a serum albumin shell (e.g., OPTISON®), wherein the microbubbles are associated a vitreolytic agent, such as a liquefactant or interfactant.

The composition may comprise microbubbles and one or more pharmaceutically acceptable carriers, excipients or diluents. A pharmaceutically “acceptable” carrier is generally compatible with the other ingredients of the composition and not injurious to the patient. The compositions may conveniently be presented in unit dosage form and may be prepared by any methods well known in the art of pharmacy.

In certain embodiments, the composition of the present invention is suitable for injection into the eye. Injectables for such use can be prepared in conventional forms, either as a liquid solution or suspension or in a solid form suitable for preparation as a solution or suspension in a liquid prior to injection, or as an emulsion. Carriers can include, for example, water, saline (e.g., normal saline (NS), phosphate-buffered saline (PBS), balanced saline solution (BSS)), sodium lactate Ringer's solution, dextrose, glycerol, ethanol, and the like; and if desired, minor amounts of auxiliary substances, such as wetting or emulsifying agents, buffers, and the like can be added.

Proper fluidity can be maintained, for example, by using a coating such as lecithin, by maintaining the required particle size in the case of dispersion and by using surfactants.

The present invention extends to methods of preparing such microbubble compositions. comprising admixing the components of a microbubble solution and optionally, one or more therapeutic agents. In certain embodiments, an external rocking, vibrating or rotating mechanism may be added to increase mixing efficiency, without the need for an internal mixing device.

In a particular embodiment, the composition is prepared by vigorously shaking a vial of microbubble solution for 30-90 seconds on a microbubble shaker. In embodiments where enzymes are to be associated with the microbubbles, an appropriate amount of enzyme or vitrolyetic agent is diluted in balanced salt solution and combined with an appropriate amount of microbubble solution. The enzyme-microbubble mixture or vitreolytic agent-microbubble mixture is vigorously shaken for an additional 30-90 seconds on a microbubble shaker prior to administration.

Apart from mechanical agitation, other methods may be used to provide the microbubble composition of the present invention including emulsification, probe sonification, spray drying or flow focusing.

In one embodiment, the microbubbles are preparing by forming a solution comprising a stabilizing material and a core gas material in the liquid state. The solution is homogenized and shaken to form a gas-filled microbubble foam.

The stabilizing material may be any material that effects the stability of compositions containing the gases, gaseous precursors, liquids, targeting ligands and/or other therapeutically actives agents (e.g., enzymes) described herein, including, for example, mixtures, suspensions, emulsions, dispersions, vesicles, microspheres or the like. Exemplary stabilizing materials include, for example, lipids, proteins, polymers, carbohydrates and surfactants.

The improved stability involves, for example, the maintenance of a relatively balanced condition, and may be exemplified, for example, by increased resistance of the composition against destruction, decomposition, degradation, and the like. In certain embodiments, composition comprises vesicles filled with gases, gaseous precursors, liquids, target ligands and/or therapeutic agents, wherein the stabilizing compounds may serve to either form the vesicles or stabilize the vesicles, in either way serving to minimize or substantially (including completely) prevent the escape of gases, gaseous precursors and/or therapeutic agents from the vesicles until release is desired.

Methods of Use

In one aspect, the present invention is a method of treating a disease or disorder of the eye in a subject in need thereof, comprising (i) administering the composition of the present invention to a subject in need thereof and (ii) applying ultrasound to active the microbubbles, resulting in complete or partial treatment of the disease or disorder of the eye.

The ultrasound may be applied at a predetermined time or during live imaging.

As used herein, the term “treatment” or “treat” as used herein means any treatment of disease or disorder in a mammal, particularly a human, including: (a) protecting against the disease or disorder, that is, causing the clinical symptoms not to develop; (b) inhibiting the disease or disorder, that is, arresting, ameliorating, reducing, or suppressing the development of clinical symptoms; and/or (c) relieving the disease or disorder, that is, causing the regression of clinical symptoms. It will be understood by those skilled in the art that in human medicine, it is not always possible to distinguish between “preventing” and “suppressing” since the ultimate inductive event or events may be unknown, latent, or the patient is not ascertained until well after the occurrence of the event or events. Therefore, as used herein the term “prophylaxis” is intended as an element of “treatment” to encompass both “preventing” and “suppressing” as defined herein. The term “protection,” as used herein, is meant to include “prophylaxis.” The “subject” as used herein refer to any mammal, especially a human. Microbubble preparations are administered to the subject in need thereof by any method known in the art suitable to position the microbubble preparation at the intended intraocular ocular site. Non-limiting example of methods for delivery to the intended ocular region include administration of the microbubble composition by direct injection into the eye (vitreous cavity, subretinal or anterior chamber) and intravascular administration. In one embodiment, the method of administration is by direct injection into the eye, performed using the pars plana route with the needle directed towards the midvitreous cavity.

Administration may also be topical. For example, solutions or suspensions of the compound or compositions of the invention may be formulated as eye drops, or as a membranous ocular patch, which is applied directly to the surface of the eye.

The method may involve a delivery device, for example an invasive or non-invasive ocular delivery device.

In different embodiments, the enzyme or vitreolytic agent can be associated with the microbubbles or co-administered as a mixture with microbubbles prior to providing ultrasound energy to activate the microbubbles and treat a tractional membrane.

In one embodiment, the present invention is a method of treating a disease or disorder of the eye in a subject in need thereof, comprising (i) administering a composition comprising microbubbles, wherein the microbubbles are optionally associated an enzyme or vitreolytic agent and (ii) applying ultrasound to activate the microbubbles to achieve complete or partial treatment of the disease or disorder of the eye.

In a particular embodiment, the present invention is a method of treating tractional membrane in the eye in a subject in need thereof, comprising (i) administering a composition comprising microbubbles, wherein the microbubbles are optionally associated an enzyme or vitreolytic agent and (ii) applying ultrasound to activate the microbubbles to achieve complete or partial treatment the tractional membrane.

In another particular embodiment, the present invention is a method of treating a macular hole in the eye in a subject in need thereof, comprising (i) administering a composition comprising microbubbles, wherein the microbubbles are optionally associated an enzyme or vitreolytic agent and (ii) applying ultrasound to activate the microbubbles to achieve complete or partial treatment of the macular hole.

In a particular embodiment, treatment includes detachment of the vitreous and/or reducing or relieving tension on tractional membranes

In a particular embodiment, the present invention is a method of treating tractional membranes in a subject in need thereof, comprising (i) administering a composition comprising microbubbles by intravascular administration, wherein the microbubbles are optionally associated with one or more therapeutic agents and (ii) applying ultrasound to activate the microbubbles to achieve complete or partial treatment of the tractional membrane. In one embodiment, the therapeutic agent is an enzyme, such as a proteolytic enzyme. In another embodiment, the therapeutic agent is a vitreolytic agent, such as an enzymatic vitreolytic agent or a non-enzymatic vitreolytic agent.

In another particular embodiment, the present invention is a method of treating tractional membranes in a subject in need thereof, comprising (i) administering a composition comprising microbubbles by direct injection into the eye vitreous cavity, subretinal or anterior chamber, wherein the microbubbles are optionally associated with one or more therapeutic agents and (ii) applying ultrasound to activate the microbubbles to achieve complete or partial treatment. In a particular embodiment, treatment includes detachment of the vitreous and/or reducing or relieving tension on tractional membranes. In one embodiment, the therapeutic agent is an enzyme, such as a proteolytic enzyme. In another embodiment, the therapeutic agent is a vitreolytic agent, such as an enzymatic vitreolytic agent or a non-enzymatic vitreolytic agent.

In another particular embodiment, the present invention is a method of treating a macular hole in a subject in need thereof, comprising (i) administering a composition comprising microbubbles by intravascular administration, wherein the microbubbles are optionally associated with one or more therapeutic agents and (ii) applying ultrasound to activate the microbubbles to achieve complete or partial treatment of the macular hole. In one embodiment, the therapeutic agent is an enzyme, such as a proteolytic enzyme. In another embodiment, the therapeutic agent is a vitreolytic agent, such as an enzymatic vitreolytic agent or a non-enzymatic vitreolytic agent.

In another particular embodiment, the present invention is a method of treating a macular hole in a subject in need thereof, comprising (i) administering a composition comprising microbubbles by direct injection into the eye vitreous cavity, subretinal or anterior chamber, wherein the microbubbles are optionally associated with one or more therapeutic agents and (ii) applying ultrasound to activate the microbubbles to achieve complete or partial treatment of the macular hole. In another embodiment, the therapeutic agent is a vitreolytic agent, such as an enzymatic vitreolytic agent or a non-enzymatic vitreolytic agent.

Ultrasound techniques can be used to image or visualize the microbubbles at the desired location in the patient, and also to activate microbubbles for therapeutic applications.

Ultrasonic techniques have also been utilized in surgical procedures on the eye for imaging structure and/or tissue of a surgical site. See, e.g., U.S. Pat. No. 6,676,607 to de Juan, Jr. et al., the contents of which are incorporated herein by reference in their entirety. In one embodiment, topical ultrasound gel is applied to the ocular surface. In another embodiment an ultrasound probe is applied to the inferior conjuctival surface aiming at the midvitreous cavity.

Ultrasound energy is applied in an amount sufficient to cause the microbubbles to activate or rupture. The ultrasound energy can be applied externally from outside the eye globe or internally from inside the eye. The treatment can be directed at the cornea, the front most layer of the eye. The treatment enhances therapy of corneal disease.

In one embodiment, the ultrasound mechanical index used is less than about 0.50, between about 0.01 and 0.50, or between about 0.20 and 0.32. In one embodiment, the ultrasound mechanical index used is between about 0.25 and 0.32. In one particular embodiment, the ultrasound mechanical index used is about 0.25. In another particular embodiment, the ultrasound mechanical index used is about 0.29.

In one embodiment, the ultrasound frequency is between about 1-20 MHz, between about 1-10 MHz, between about 1-5 MHz, between about 6-10 MHz , between about 4-12 MHz, between about 2-15 MHz, between about 10-25 MHz or between about 5-15 MHz. In another embodiment, the ultrasound frequency is about 1 MHz, about 2 MHz, about 3 MHz, about 4 MHz, about 5 MHz, about 6 MHz, about 7 MHz, about 8 MHz, about 9 MHz, about 10 MHz, about 15 MHz, or about 20 MHz. In one particular embodiment, the ultrasound frequency is about 8 MHz.

In one embodiment, the ultrasound application time is between about 1-60 minutes, between about 20-40 minutes, between about 10-45 minutes. In one particular embodiment, the ultrasound application time is about 20 about minutes, 25 about minutes, 30 about minutes, 35 about minutes, 40 about minutes, or 45 about minutes.

In one particular embodiment, the ultrasound mechanical index is 0.25, the frequency is 8 MHz and the application time is 30 minutes.

In one embodiment, the present invention is a method of treating tractional membranes in a subject in need thereof, comprising (i) administering a composition comprising microbubbles by a method selected from intravascular administration or direct injection to the eye, wherein the microbubbles are optionally associated with one or more therapeutic agents selected from the group consisting of enzymes and vitreolytic agents and (ii) applying ultrasound to the external service of the eye to activate the microbubbles, thereby achieving complete or partial treatment of the tractional membrane. In a particular embodiment, treatment includes detachment of the vitreous and/or reducing or relieving tension on tractional membranes. Optionally, the method further comprising visualizing the microbubbles before applying ultrasound.

In another embodiment, the present invention is a method of treating tractional membranes in a subject in need thereof, comprising (i) administering a composition comprising microbubbles by intravascular administration or direct injection to the eye, wherein the microbubbles are optionally associated with one or more therapeutic agents selected from the group consisting of enzymes and vitreolytic agents and (ii) applying intraocular ultrasound to activate the microbubbles, thereby achieving complete or partial treatment of the tractional membrane. In a particular embodiment, treatment includes detachment of the vitreous and/or reducing or relieving tension on tractional membranes. Optionally, the method further comprising visualizing the microbubbles before applying ultrasound.

In a further embodiment, the present invention is a method of treating tractional membranes in a subject in need thereof, comprising (i) administering a composition comprising microbubbles by a method selected from intravascular administration or direct injection to the eye, wherein the microbubbles are associated with one or more therapeutic agents selected from the group consisting of enzymes and vitreolytic agents and (ii) applying ultrasound to the external surface of the eye to activate the microbubbles, thereby releasing the therapeutic agent and achieving complete or partial treatment of the tractional membrane. In a particular embodiment, treatment includes detachment of the vitreous and/or reducing or relieving tension on tractional membranes. Optionally, the method further comprising visualizing the microbubbles before applying ultrasound.

In a still further embodiment, the present invention is a method of treating tractional membranes a subject in need thereof, comprising (i) administering a composition comprising microbubbles by intravascular administration or direct injection to the eye, wherein the microbubbles are associated with one or more therapeutic agents selected from the group consisting of enzymes and vitreolytic agents and (ii) applying intraocular ultrasound to activate microbubbles to release therapeutic agents, thereby achieving complete or partial treatment of the tractional membrane.

In a particular embodiment, treatment includes detachment of the vitreous and/or reducing or relieving tension on tractional membranes. Optionally, the method further comprising visualizing the microbubbles before applying ultrasound.

In a particular embodiment, the present invention is a method of treating tractional membranes in a subject in need thereof, comprising (i) administering a composition comprising microbubbles by a method selected from intravascular administration or direct injection to the eye, wherein the microbubbles are associated with an enzyme selected from the group consisting of chondroitinase, collagenase, dispase, hyaluronidase, nattokinase, plasmin, plasminogen activators and ocriplasmin (microplasmin), alone or in combination, and (ii) applying ultrasound to the external surface of the eye to activate the microbubbles, thereby releasing the therapeutic agent and achieving complete or partial treatment of the tractional membrane. In a particular embodiment, treatment includes detachment of the vitreous and/or reducing or relieving tension on tractional membranes. Optionally, the method further comprising visualizing the microbubbles before applying ultrasound.

In a specific embodiment, the present invention is a method of treating tractional membranes in a subject in need thereof, comprising (i) administering a composition comprising microbubbles by a method selected from intravascular administration or direct injection to the eye, wherein the microbubbles are associated with microplasmin, alone or in combination with another therapeutic agent(s), and (ii) applying ultrasound to the external service of the eye to activate the microbubbles, thereby releasing the microplasmin and optionally, the one or more additional therapeutic agents, to achieve complete or partial treatment of the tractional membrane. In a particular embodiment, treatment includes detachment of the vitreous and/or reducing or relieving tension on tractional membranes. Optionally, the method further comprising visualizing the microbubbles before applying ultrasound.

In another specific embodiment, the present invention is a method treating tractional membranes in a subject in need thereof, comprising (i) administering a composition comprising microbubbles by intravascular administration or direct injection to the eye, wherein the microbubbles are associated with microplasmin, alone or in combination with another therapeutic agent(s) and (ii) applying intraocular ultrasound to activate microbubbles to release therapeutic agents and thereby achieve complete or partial treatment. In a particular embodiment, treatment includes detachment of the vitreous and/or reducing or relieving tension on fractional membranes. Optionally, the method further comprising visualizing the microbubbles before applying ultrasound.

In one embodiment, the present invention is a method of achieving detachment of the vitreous and/or reducing or relieving tension on tractional membranes in the eye in a subject in need thereof by (i) administering a composition comprising microbubbles, wherein the microbubbles are optionally associated (e.g., coated or filled with) an enzyme (e.g., proteolytic enzyme) or vitreolytic agent, and (ii) applying ultrasound to active the microbubble, thereby releasing the enzyme and achieving detachment of the vitreous and/or reducing or relieving tension on tractional membranes. In a particular embodiment, the microbubbles are administered to the eye vitreous cavity, subretinal or anterior chamber of the eye.

In a particular embodiment, the present invention is a method of achieving detachment of the vitreous and/or reducing or relieving tension on tractional membranes in the eye in a subject in need thereof by (i) administering a composition comprising microbubbles, wherein the microbubbles are associated (e.g., coated or filled) with an enzyme selected from the group consisting of chondroitinase, collagenase, dispase, hyaluronidase, nattokinase, plasmin, plasminogen activators and ocriplasmin (microplasmin), alone or in combination, and (ii) applying ultrasound to active the microbubble, thereby releasing the enzyme and achieving detachment of the vitreous and/or reducing or relieving tension on tractional membranes.

In a particular embodiment, the present invention is a method of achieving detachment of the vitreous and/or reducing or relieving tension on tractional membranes in the eye in a subject in need thereof by (i) administering a composition comprising microbubbles, wherein the microbubbles are associated (e.g., coated or filled) with microplasmin alone or in combination with one or more therapeutic agents, and (ii) applying ultrasound to active the microbubble, thereby releasing the enzyme and the additional therapeutic agent(s), if any, and achieving detachment of the vitreous and/or reducing or relieving tension on tractional membranes.

In another embodiment, the present invention is a method of inducing pharmacologic vitreolysis, in the eye in a subject in need thereof by (i) administering a composition comprising microbubbles, wherein the microbubbles are associated (e.g., coated or filled with) an enzyme (e.g., a proteolytic enzyme) or a vitreolytic agent and (ii) applying ultrasound to active the microbubble, thereby releasing the enzyme and achieving complete or partial pharmacologic vitreolysis.

In a particular embodiment, the present invention is a method of achieving pharmacologic vitreolysis a subject in need thereof by (i) administering a composition comprising microbubbles, wherein the microbubbles are associated (e.g., coated or filled) with an enzyme selected from the group consisting of chondroitinase, collagenase, dispase, hyaluronidase, nattokinase, plasmin, plasminogen activators and ocriplasmin (microplasmin), alone or in combination, and (ii) applying ultrasound to active the microbubble, thereby releasing the enzyme and achieving complete or partial pharmacologic vitreolysis.

In a particular embodiment, the present invention is a method of achieving pharmacologic vitreolysis in a subject in need thereof by (i) administering a composition comprising microbubbles, wherein the microbubbles are associated (e.g., coated or filled) with microplasmin alone or in combination with one or more therapeutic agents, and (ii) applying ultrasound to active the microbubble, thereby releasing the enzyme and the additional therapeutic agent(s), if any, thereby and achieving complete or partial pharmacologic vitreolysis.

Other therapeutic agents suitable for use in the method of the present invention include, but are not limited to, anticoagulants, antithrombolytic agent, antiinflammatory agenst, antiviral agents, antibacterial agents, antifungal agents, anti-angiogenic agent, anti-mitotic agents, antihistamines or anesthetics. The therapeutic agent may be, for example, and inorganic compound, biological polymer, peptide, polypeptide, antibody, peptide conjugate, nucleic acid, oligonucleotide, polynucleotide, ribozyme, or small interfering RNA (siRNA) capable of rendering a beneficial physiological effect in treating a pathological condition.

Endpoints to evaluate efficacy of treatment according to the methods of the present invention include, but are not limited to complete vitreous detachment, resolution of macular hole, and improved best-corrected visual acuity by 3 or more lines.

Treatment according to the methods of the present invention may include, for example, inducing posterior vitreous detachment (PVD) in the eye, liquefaction of the vitreous in the eye, posterior vitreous detachment, resolving vitreomacular adhesion or closing macular holes.

In one embodiment, the method of the present invention is useful for inducing an innocuous (and not anomalous) PVD.

The subject in need thereof may be otherwise healthy or suffering for an underlying disease or disorder, including an underlying disease or disorder of the eye or that manifests ocular symptoms other than tractional membranes.

The method of the present invention is useful for treating vitreo-pathologies, such as vitreo-retinopathologies and vitreo-macularpathologies.

In one embodiment, the method of the present invention is useful to treat a disease or disorder selected from the group consisting of rhegmatogenous retinal detachment, advanced diabetic retinopathy, macular pucker, macular holes and wet age-related macular degeneration.

In a particular embodiment, the method of the present invention is useful to treat a disease or disorder selected from the group consisting of myopic vitreopathy and diabetic vitreopathy.

In one embodiment, the microbubble composition administered comprises microbubbles unassociated with a therapeutic agent or more specifically, unassociated with an enzyme or proteolytic enzyme. The microbubbles are injected into the vitreous and agitated with extraocular ultrasound (with or without cavitation) to effect treatment of vitreomacular traction and/or vitreous detachment. In a particular embodiment, the microbubbles are commercially available gas-filled DEFINITY™ microbubbles.

In a particular embodiment, the microbubble composition administered comprises microbubbles associated with a therapeutic agent, such as an enzyme or vitreolytic agent. In one embodiment, the microbubbles contain the enzyme or vitreolytic agent. In another embodiment, the enzyme or vitreolytic agent is coated on the surface of the microbubbles or otherwise associated with the microbubble surface.

In a particular embodiment, the microbubble composition comprises microbubbles associated with plasmin or microplasmin or hyaluronidase.

In certain embodiments, the microbubble composition is injected into the vitreous and the microbubbles are agitated with extraocular ultrasound (with or without cavitation) to effect treatment of vitreomacular traction and/or effect vitreous liquefaction and its detachment from the macula.

In certain embodiments, the microbubble composition is administered together with a therapeutic agent, such as an enzyme or vitreolytic enzyme, but the microbubbles are not associated with the enzyme, i.e., the thereapeutic agent is not directly associated with the microbubbles. In this embodiment, the microbubble composition and the therapeutic agent are administered to the patient. The microbubble composition and the therapeutic agent (e.g., the enzyme or vitreolytic agent) may be administered in any order, including concurrently.

In one embodiment, activation of the microbubbles with ultrasound releases the therapeutic agent substantially uniformly or homogenously throughout the vitreous so as to uniformly contact the intravitreal membrane.

In one embodiment, the microbubble composition is applied to the surface(s) of the eye that is/are loaded with therapeutic agents and the ultrasound treatment facilitates entry of the drug(s) into the anterior chamber of the eye, delivering therapeutics to the interior of the eye.

The composition of the present invention is administered to the subject in a therapeutically effective amount administered to the patient in a therapeutically effective amount. As used herein, a therapeutically effective amount is intended to include at least partially attaining the desired effect, or delaying the onset of, or inhibiting the progression of, or halting or reversing altogether the onset or progression of the disease or disorder.

In a particular embodiment, the therapeutically effective amount is that a dose that can effect clinical benefit such as inducing posterior vitreous detachment without significant side effects such as retinal detachment.

In one embodiment, the microbubble composition is administered in a dose that provides a dose of the enzyme or vitreolytic agent (w/v) of between about 0.1-500 μg, between about 0.1-100 μg, about 0.1-300 μg, between about 10-300 μg, between about 50-200 μg, between about 80-150 μg, between about 80-120 μg, between about 1-50 μg between about 10-40 μg, between about 20-30 μg, between about 1-30 μg between about 30-50 μg, between about 50-80 μg, between about 20-80 μg, between about 30-70 μg, between about 40-60 μg, about 5 μg, about 10 μg, about 20 μg, about 30 μg, about 40 μg, about 50 μg, about 60 μg, about 70 μg, about 80 μg, about 90 μg, about 100 μg, about 110 μg, about 120 μg, about 130 μg, about 140 μg, or about 150 μg of enzyme.

In another embodiment, the enzyme or vitreolytic agent is administered in a dose of between about 0.1-500 μg, between about 0.1-100 μg, about 0.1-300 μg, between about 10-300 μg, between about 50-200 μg, between about 80-150 μg, between about 80-120 μg, between about 1-50 μg between about 10-40 μg, between about 20-30 μg, between about 1-30 μg between about 30-50 μg, between about 50-80 μg, between about 20-80 μg, between about 30-70 μg, between about 40-60 μg, about 5 μg, about 10 μg, about 20 μg, about 30 μg, about 40 μg, about 50 μg, about 60 μg, about 70 μg, about 80 μg, about 90 μg, about 100 μg, about 110 μg, about 120 μg, about 130 μg, about 140 μg, or about 150μg of enzyme.

The dose may be administered in a single dose or a series of doses.

In a particular embodiment, the enzyme is microplasmin and the dose is less than the dose required to achieve pharmacologic vitreolysis when administered in the absence of the microbubble composition of the present invention.

In some embodiments, the method may serve as a treatment to be repeated over time. For example, over a period of days, weeks, months or years.

### EXAMPLES

### Example 1

### Evaluating Efficacy of Plasmin-Microbubble Ultrasound on Inducing Posterior Vitreous Detachment (PVD)

Animal experiments were performed at the facilities of Doheny Eye Institute on a population of 20 normal age-matched rabbits assigned to 4 experimental groups. The study rationale was to evaluate the possibility of induction of a posterior vitreous detachment (PVD) using a reduced dose of plasmin with the advantage of microbubble-assisted ultrasound as a novel mode of ocular delivery.

Eyes were submitted to histopathology including H&E slides, scanning electron microscopy, and immunostaining studies after completion of the study.

Experimental Groups Group-A (n=5): received intravitreal injection of 100 μl microbubble solution (DEFINITY™) followed by 30 minutes of ultrasound application.

Group-B (n=5): received intravitreal injection of 100 μl of plasmin (100 μg).

Group-C (n=5): received intravitreal injection of 100 μl of full-dose plasmin (500 μg).

Group-D (n=5): received intravitreal injection of 50 μl of the plasmin (50 μg)+50 μl MB mixture followed by 30 minutes of ultrasound application.

Plasmin-Microbubble Preparation
DEFINITY™ Microbubble vial is placed over the Microbubble shaker and shaken vigorously for 45 seconds
25 μL Microplasmin is diluted with 25 μL balanced salt solution (BSS) to get a concentration of 1:2
50 μL DEFINITY™ Microbubbles is added to 50 μL of the previous concentration to get a final Microplasmin concentration of 1:4
Microplasmin-Microbubble admixture will be vigorously shaken for another 45 seconds

Intravitreal Injections
Intravitreal injection of 100 μL of the previous concentration using 33-G needle connected to 100 μL, Hamilton syringe
Injection will be performed using the pars plana route with the needle directed towards the midvitreous cavity
Intravitreal injections were performed in sterile conditions and after instillation of several drops of 5% Povidone iodine. Topical tetracaine drops were applied for additional anesthesia. A speculum was used to keep the eyelids open, and the procedure was performed under direct visualization using an operating microscope and a surgical contact lens. A 30-gauge needle attached to a 1-mL syringe was passed into the midvitreous cavity transconjunctivally, at the upper temporal quadrant of the globe, 2 mm behind the limbus.
At the end of the procedure, topical antibiotic ointment (Neo-Poly-Bac-Bausch & Lomb Inc., Tampa, Fla.) was applied to the conjunctiva sac.
Application of topical ultrasound gel to the ocular surface

Ultrasound settings:
1. Frequency: 8 MHz
2. Mechanical index: 0.25
3. Duration: 30 minutes
4. Site of application: direct over the inferior conjunctival surface.

Follow-up:
1. Baseline fluorescein angiography (FA) and optical coherence tomography (OCT) was performed at initial visit and again at 1 and 2 weeks after the procedure.
2. Follow-up imaging: every 5 days to a total of 3 visits.

Ocular Examination Examinations included evaluation of the anterior segment by slit lamp (Haag-Streit Diagnostics, Gartenstadtstrasse, Switzerland), fluorescein angiography (FA), and optical coherence tomography (OCT). Before each session, the rabbits were anesthetized, and the pupils were dilated as described earlier. Baseline examinations were performed right away before the laser procedure, and follow-up examinations were performed every two weeks after the primary procedure till the end of the eight-week-follow-up period.

Optical Coherence Tomography The Spectralis HRA-OCT (Heidelberg Engineering GmbH, Heidelberg, Germany) is a high-resolution spectral domain OCT that can simultaneously perform scanning laser ophthalmoscopy. High-resolution horizontal raster 6×6-mm scans through the laser-treated lesions. All of the scans were performed using the eye-tracking system. Only the scans that were of sufficient or good quality (>20 dB) and fulfilled OSCAR-IB quality control criteria for OCT scans were included in the study results. The lesion site was examined by OCT, using the single line or raster scan modes. The OCT evaluation of the lesion site was used to identify the presence of reflections in the subretinal space for correlation with histologic data.

Fluorescein Angiography The same camera system used for OCT was set for FA by selecting the appropriate settings and filter. An intravenous line was established on the marginal ear vein and 0.2 mL of AK-Fluor® (fluorescein injection 10%-Akorn, Abit Springs, La.) was injected and flushed with 1 mL of normal saline. Sequential fundus photographs were taken immediately after fluorescein injection. Late photographs were taken at 3 and 5 minutes.

Euthanasia At the end of the last follow-up examination in each group, the rabbits were euthanized by intracardiac injection of 2 mL pentobarbital (Beuthanasia-D; Schering Plough Animal Health, Omaha, Nebr.).

Light Microscopic Examination Enucleated eyes were immersed in Davidson's fixative solution (two eyes from each group) overnight and then dehydrated in a series of graded alcohol solutions over the next 24 to 48 hours before paraffin embedding. Blocks were obtained from cuts through the whole globe oriented perpendicularly to the medullary wings. Sections 5-μm thick were obtained by microtome, stained with hematoxylin and eosin (H&E) stains, and examined by light microscopy. Retinal regions 4 disc diameters below the optic disc were photographed at the same magnification (Photoshop, version CS4; Adobe, Mountain View, Calif.).

Scanning Electron Microscopy For this purpose, 5×5-mm ocular samples (4 disc diameters below the optic disc) were sectioned from the enucleated eyes after stripping of the neurosensory retina to be fixed in half-strength Karnovsky's fixative for 2 days. After initial fixation, the specimens were rinsed several times with phosphate buffer then post fixed with 1% osmium tetroxide in 0.1 M PBS for 1 hour. After a rinse with PBS, the specimens were dehydrated using a series of graded ethyl alcohol solutions and then chemically dried using hexamethyldisilazane. After drying, the specimens were mounted on aluminum stubs with adhesive tabs and sputter coated for 3 minutes (Polaron; Energy Beam Sciences, Agawam, Mass.). Specimens were then viewed under a scanning electron microscope (JSM-6390-JEOL USA, Inc., Peabody, Mass.).

Results:

Treatment groups were compared regarding the success rate of inducing PVD and the incidence of any complications, specifically retinal detachment. Group-A receiving vehicle and ultrasound only, showed no success in inducing PVD and no incidence of retinal detachment. Group B received ¼ dose of plasmin without ultrasound showed no success in inducing PVD and no incidence of retinal detachment. Group-C having the full-dose of plasmin without ultrasound showed a 100% success rate in inducing PVD, but had a high incidence (80%) of retinal detachment in the group. Group-D received a ¼^{th} dose of plasmin with microbubble vehicle and ultrasound and similarly showed 100% success in inducing PVD and 0% retinal detachment.

The lower dose of plasmin in the presence of ultrasound shows similar efficacy and better safety profile shown by the absence of retinal detachment after plasmin/microbubble-ultrasound therapy.

All patents and patent publications referred to herein are hereby incorporated by reference.

While certain embodiments have been described herein, it will be understood by one skilled in the art that the methods, systems, and apparatus of the present disclosure may be embodied in other specific forms without departing from the spirit thereof.

We claim:

1. A composition for comprising microbubbles associated one or more therapeutic agents selected from the group consisting of enzymes and non-enzymatic vitreolytic agents.

2. The composition of claim 1, wherein the enzyme selected from the group consisting of chondroitinase, collagenase, dispase, hyaluronidase, nattokinase, plasmin, plasminogen activators and microplasmin.

3. The composition of claim 1, wherein the enzyme is a plasmin, microplasmin or hyaluronidase.

4. The composition of claim 1, wherein the enzyme is microplasmin.

5. The composition of claims 1, wherein the composition is formulated for intraocular delivery.

6. The composition of claims 1 wherein the microbubbles are gas-filled lipid or protein microbubbles.

7. The composition of claim 1, wherein the microbubbles are octafluoropropane-filled lipid microbubbles.

8. A method of treating a tractional membrane of the eye in a subject in need thereof comprising: (i) administering an effective amount of a composition comprising microbubbles to the tractional membrane of the eye in need of treatment, wherein the microbubbles are associated with one or more therapeutic agents selected from the group consisting of enzymes and non-enzymatic vitreolytic agents and (ii) applying ultrasound energy generated by an ultrasound probe to the eye, wherein the application of ultrasound energy is sufficient to activate the microbubble composition and treat the tractional membrane.

9. The method of claim 8, wherein the microbubbles are associated with an enzyme.

10. The method of claim 8, wherein the microbubbles are associated with a non-enzymatic vitreolytic agent.

11. The method of claim 8, wherein the microbubble are coated or filled with the therapeutic agent.

12. The method of claim 9, wherein the enzyme is a proteolytic enzyme.

13. The method of claim 9, wherein the enzyme is administered at a dose of between about 10 μg and about 300 μg.

14. The method of claim 13, wherein the dose is about 100 μg.

15. The method of claim 13, wherein the dose is about 50 μg.

16. The method of claim 8, wherein ultrasound is applied to the inferior conjunctival surface.

17. The method of claim 8, wherein the ultrasound frequency of between about 1-20 MHz.

18. The method of claim 8, wherein the ultrasound frequency of between about 1-10 MHz.

19. The method of claim 8, wherein the ultrasound mechanical index is between about 0.01 and about 0.5.

20. The method of claim 8, wherein the ultrasound frequency is about 8 MHz and the ultrasound mechanical index is about 0.25.

21. The method of claim 8, wherein activation of the microbubble composition effects vitreolysis.

22. The methods of claim 21, wherein the vitreolysis results in PVD.

23. The method of claim 8, wherein activation of the microbubble composition effects vitreolysis without retinal detachment.
